(19) United States
(12) Patent Application Publication
(10) Pub. No.: US 2005/0210963 A1
(43) Pub. Date: Sep. 29, 2005

(54) Fluid monitoring and sampling apparatus

(76) Inventors: David Yekutiely, Ramat HaSharon (IL); Barak Yekutiely, Kochav Yair (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/091,582

(22) Filed: Mar. 29, 2005

Related U.S. Application Data
(60) Provisional application No. 60/556,880, filed on Mar. 29, 2004.

(51) Int. Cl. G01N 33/00; G01F 23/36; G01N 9/12; G01N 1/10

(52) U.S. Cl. 73/53.01; 73/64.56; 73/453; 73/306

(57) ABSTRACT

Fluid monitoring and sampling apparatus including a buoyant element suspended from a filament, a load sensor that senses a tensile force in the filament, a rotation sensor that senses the rotation of the spool around which the filament is wound, wherein the buoyant element is adapted to be initially at least partially submerged at an equilibrium position at an initial level of a fluid, thereby creating a nominal tensile force in the filament, wherein a change in the level of the fluid changes the tensile force in the filament, a positive change in the tensile force corresponding to a downward movement of the buoyant element and a negative change in the tensile force corresponding to an upward movement of the buoyant element, wherein the rotation of the spool corresponds to an amount of distance traveled by the buoyant element, and a sensor for sensing a property of the fluid, the sensor being in communication with a processor above ground.

### FIELD OF THE INVENTION

The present invention relates generally to fluid level gauges or monitors, and particularly to a fluid level gauge or monitor for use with water or fuel wells, and the like.

### BACKGROUND OF THE INVENTION

In many localities, water is supplied to consumers by pumping the water from wells. Water wells can be quite deep, some reaching depths of over 500 meters. In states or countries that have low amounts of precipitation, well water is a precious commodity, and wells are intensively pumped to meet the consumer demand. In such cases, the level of the water in the well can reach low levels, and the pumped water can become mixed with sand or seawater. It is readily understood that such a situation is undesirable and intolerable. The sand that is pumped with the water can foul and damage irrigation pumps of agricultural consumers. The quality of water mixed with seawater is intolerable and dangerous for drinking purposes. It is thus imperative to monitor the water level in the well, in order to know when to stop pumping water from the well. Unfortunately, the prior art has no known solution for real-time monitoring of water level in a well, especially deep wells.

### SUMMARY OF THE INVENTION

The present invention seeks to provide a novel fluid level monitor (or gauge, the terms being used interchangeably herein) that can be used for real-time monitoring of water level and properties in a well and the like, as is described more in detail hereinbelow. Although the present invention is described herein for water wells, nevertheless the invention is applicable for any kind of fluid, such as oil.

The present invention may include a system built similarly to that described in U.S. Pat. No. 6,508,120 to the same inventors, but with added features as is described more in detail hereinbelow.

### BRIEF DESCRIPTION OF THE DRAWINGS

The present invention will be understood and appreciated more fully from the following detailed description, taken in conjunction with the drawings in which:

FIG. 1 is a simplified pictorial, partially cutaway illustration of a fluid level monitor constructed and operative in accordance with a preferred embodiment of the present invention;

FIGS. 2A-2C are simplified pictorial illustrations of operation of the fluid level monitor of FIG. 1, wherein FIG. 2A illustrates a buoyant element of the fluid level monitor at an initial, equilibrium position in a fluid, FIG. 2B illustrates the buoyant element out of the fluid, and FIG. 2C illustrates the buoyant element over-submerged in the fluid; and

FIG. 3 is a simplified illustration of a system of fluid level monitors for monitoring a plurality of wells, constructed and operative in accordance with a preferred embodiment of the present invention.

### DETAILED DESCRIPTION OF A PREFERRED EMBODIMENT

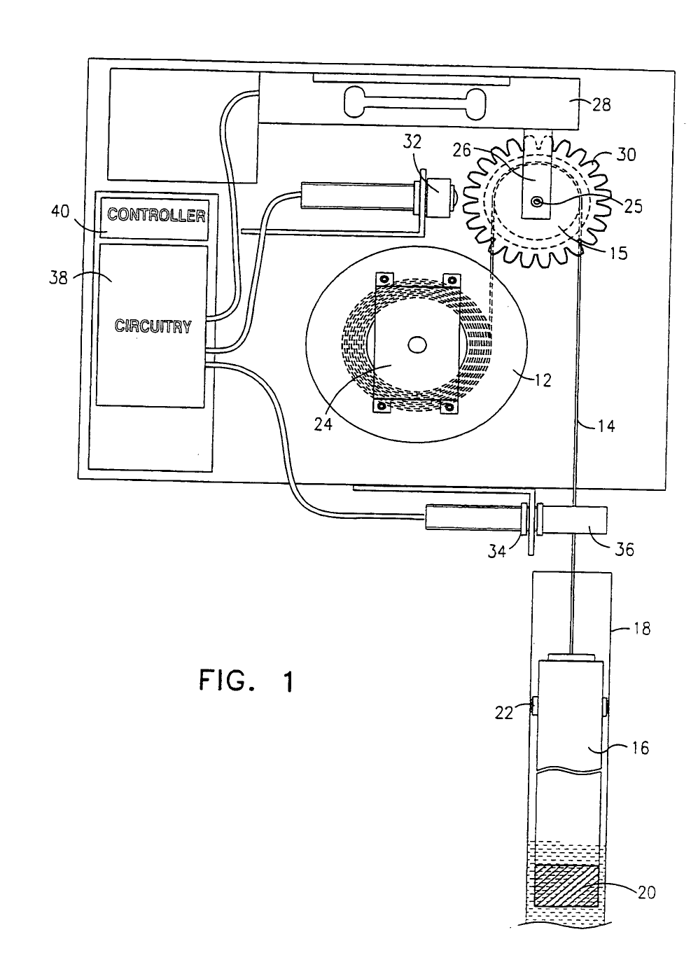

Reference is now made to FIG. 1, which illustrates fluid level monitor 10 constructed and operative in accordance with a preferred embodiment of the present invention.

Fluid level monitor 10 preferably includes a spool 12 of a filament 14. The term “filament” encompasses any string, thread, fishing line, cord, wire or rope and the like. Filament 14 is preferably wrapped one or more times around a bobbin 15, and an end of filament 14 is attached to a buoyant element 16. Buoyant element 16 is preferably disposed inside a generally vertical elongate tube 18. Such a tube is generally installed in most water wells for testing and sampling purposes, and runs virtually the entire depth of the well. The present invention exploits the fact that such a tube is present in water wells, and that such a tube offers a clean, generally undisturbed environment for buoyant element 16.

Buoyant element 16 may be fashioned in the form of a generally hollow cylinder with a weight 20 disposed at the bottom thereof (Weight 20 may fill some or all of the internal volume of buoyant element 16.) It is appreciated, however, that the invention is not limited to such a cylindrical shape, and buoyant element 16 may have any other suitable shape. In accordance with a preferred embodiment of the present invention, there are one or more friction-reducing members 22, such as rollers or low-friction pads, mounted on an external surface of buoyant element 16. Friction-reducing members 22 help ensure smooth travel of buoyant element 16 inside tube 18, and prevent buoyant element 16 from getting snagged or caught in tube 18.

Spool 12 is preferably rotated by means of a motor 24 attached thereto. Motor 24 may be a compact servomotor, for example, mounted on a central shaft of spool 12. Rotation of spool 12 either raises or lowers buoyant element 16. Bobbin 15 is preferably supported by bearings 25 mounted in a support member 26 that is attached to a load sensor 28. Load sensor 28 may be a load cell, strain or tension gauge, which can sense upward or downward flexure or movement of support member 26 (and with it upward or downward movement of buoyant element 16).

A toothed disc 30, such as a gear, is preferably coaxially mounted with bobbin 15. A proximity sensor 32 is preferably mounted in proximity to teeth 31 of disc 30. Proximity sensor 32 is preferably an induction sensor, but can also be a capacitance sensor. The assembly of spool 12, motor 24, bobbin 15, disc 30, load sensor 28 and proximity sensor 32 is preferably mounted in a housing 33. A second proximity sensor 34 is preferably mounted on a bracket 36 near an entrance/exit of filament 14 to housing 33.

Load sensor 28, motor 24 and proximity sensors 32 and 34 are preferably in electrical communication with circuitry 38 of an electronic controller 40. Circuitry 38 preferably includes any components typically used for operating the above-named parts, such as motor controls or solid state relays and the like, as is well known to the skilled artisan.

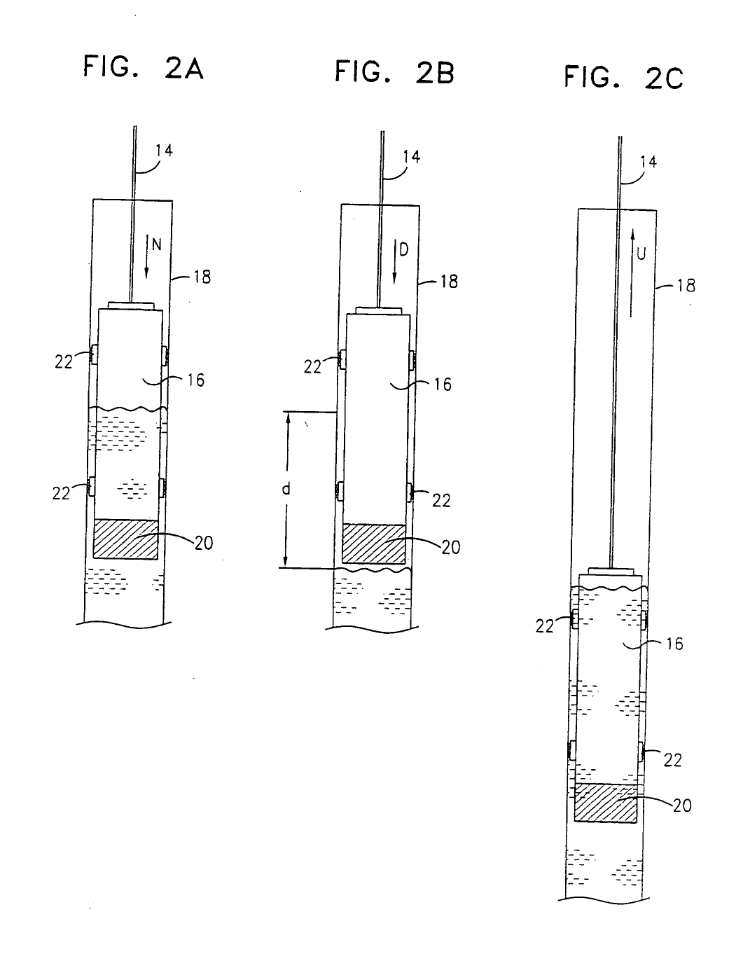

The operation of fluid level monitor 10 is now described with further reference to FIGS. 2A-2C. Buoyant element 16 partially floats at an initial level of water in tube 18, as seen in FIG. 2A. Buoyant element 16 and filament 14 are in equilibrium, i.e., buoyant element 16 has reached substantially a stable position in the water, and there is a nominal tensile force N in filament 14 due to the partially submerged weight of buoyant element 16. Nominal tensile force N is taken as the zero reference value. If the water level drops a distance d, buoyant element 16 is no longer in the water, as seen in FIG. 2B. The out-of-water weight of buoyant element 16 imparts a downward tensile force D on filament 14. Force D is transferred to and sensed by load sensor 28 as being greater than force N. This information is sent to controller 40, which understands the information to mean that force D is a downward force. Thus by comparing the sensed tension to the nominal tension in filament 14, load sensor 28 and controller 40 sense the direction of the movement of buoyant element 16. It is noted that it is not necessary for load sensor 28 to measure the exact magnitude of force D. Instead, it is sufficient to know that force D is greater than force N.

Controller 40 thereupon signals motor 24 to rotate spool 12 in a counterclockwise direction in the sense of FIG. 1, thereby spooling out filament 14 from spool 12. Bobbin 15 also turns counterclockwise, and buoyant element 16 descends into the water. As bobbin 15 turns, proximity sensor 32 counts the number of teeth 31 that pass thereby. The number of teeth 31 is interpreted and converted by controller 40 into the distance that buoyant element 16 has traveled. Proximity sensor 32 and toothed disc 30 thus act as a rotation sensor. (Although other devices, such as a shaft encoder, could be used for this purpose, the structure of the present invention is significantly simpler and less expensive.) It is appreciated that the rotation of spool 12 can be sensed, instead of that of bobbin 15. Combined with the force direction as sensed by load sensor 28, controller 40 knows the distance buoyant element 16 has traveled and in what direction.

Buoyant element 16 descends into the water to the position shown in FIG. 2C. It is seen that buoyant element 16 has “overshot” its equilibrium floating position, and is now over-submerged beyond its equilibrium point in the water. The submergence of buoyant element 16 causes filament 14 to be in less tension than the nominal tensile force N associated with the equilibrium position of buoyant element 16 in the water. In other words, the submergence of buoyant element 16 imparts an upward force U on filament 14. Force U is sensed by load sensor 28 as being less than force N. This information is sent to controller 40, which understands the information to mean that force U is an upward force.

Controller 40 thereupon signals motor 24 to rotate spool 12 in a clockwise direction in the sense of FIG. 1, thereby winding filament 14 onto spool 12. Bobbin 15 also turns clockwise, and buoyant element 16 ascends. As mentioned above, as bobbin 15 turns, proximity sensor 32 counts the number of teeth 31 that pass thereby. The number of teeth 31 is interpreted and converted by controller 40 into the distance that buoyant element 16 has traveled. The process of raising and lowering buoyant element 16 by means of load sensor 28 and controller 40 is repeated until buoyant element 16 is generally in its equilibrium position, i.e., the tensile force in filament 14 is equal to N. Preferably controller 40 will stop rotating spool 12 when the tensile force in filament 14 is within a certain predetermined tolerance near the value of N, or when a predetermined number of incremental direction changes have been made in a predetermined period of time. Once the equilibrium position has been reached, the distance that buoyant element 16 has traveled is reported or displayed by controller 40.

It is appreciated that the same explanation holds true, mutatis mutandis, for the situation wherein the water rises in tube 18, and buoyant element 16 accordingly rises as well.

Second proximity sensor 34 can be used to sense if the upper portion of buoyant element 16 has ascended to the level of bracket 36. Once buoyant element 16 has risen that high, second proximity sensor 34 signals controller 40 to stop movement of buoyant element 16. In this manner, buoyant element 16 is prevented from abutting against housing 33. Alternatively or additionally, bobbin 15 may be provided with a clutch or ratchet mechanism, so that bobbin 15 does not over-rotate and cause buoyant element 16 to abut against housing 33.

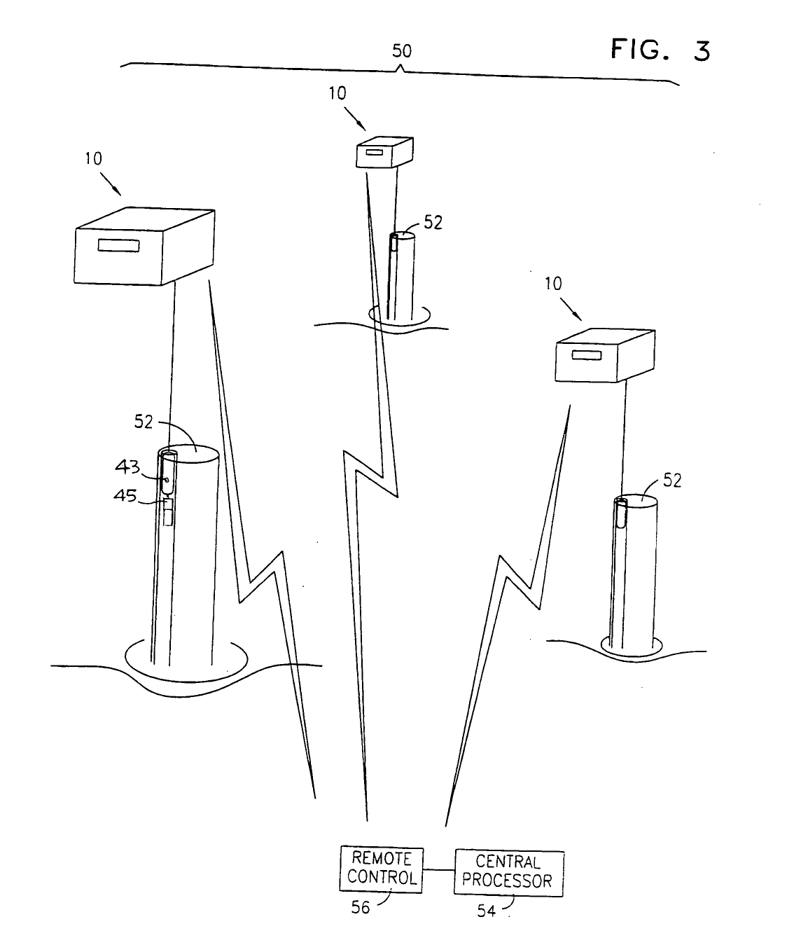

Reference is now made to FIG. 3 which illustrates a system 50 of fluid level monitors 10 for monitoring a plurality of wells 52, constructed and operative in accordance with a preferred embodiment of the present invention. System 50 preferably includes a central processor 54 in wired or wireless communication with all of the monitors 10 in the system. Monitors 10 may be remotely controlled by a remote controller 56 and/or by central processor 54 itself By using system 50, a municipality or water authority can easily monitor all of the wells in a locality or state, and can know which well is low and stop pumping supply water from that well. It is noted that in the prior art, it has not been possible to know which of the many wells (sometimes thousands) is low and is contributing to sand or sea water problems in the water supplied to consumers. With the present invention, this problem is solved.

The present invention may also be used to monitor properties of the water or other fluid, continuously in real-time along the entire depth of the well or other fluid conduit. In addition to the system described above, a sensor 43 may be provided, with or without a sample collecting vessel 45, for sensing (and sampling, if desired) fluid. The sensor 43 may sense, without limitation, the presence of dissolved solids in the fluid, the presence of oil in water, the boundary between oil and water (or other liquids) in a column of a liquid mixture, salinity, electrical conductivity, temperature, pressure, pH, viscosity, density, or any other physical, chemical, or material property. The sensor 43 may be in wireless communication with a processor (or controller, the terms being used interchangeably) above ground (e.g., central processor 54 or controller 40). The sensor 43 may be attached to (e.g., disposed in, above or below) the buoyant element 16 or weight 20. Alternatively or additionally, the sensor 43 may be spooled down separately on filament 14 and communicate wirelessly with the processor. Alternatively, filament 14 may comprise an electrical wire using single wire energy transmission techniques, as described in U.S. Pat. No. 6,1074,107, the disclosure of which is incorporated herein by reference. In such a case, the sensor 43 may be in electrical communication with the processor or controller.

1. Fluid monitoring and sampling apparatus comprising:
a buoyant element suspended from a filament;
a load sensor that senses a tensile force in said filament;
a rotation sensor that senses the rotation of said spool around which said filament is wound, wherein said buoyant element is adapted to be initially at least partially submerged at an equilibrium position at an initial level of a fluid, thereby creating a nominal tensile force in said filament, wherein a change in the level of the fluid changes the tensile force in the filament, a positive change in the tensile force corresponding to a downward movement of said buoyant element and a negative change in the tensile force corresponding to an upward movement of said buoyant element, wherein the rotation of said spool corresponds to an amount of distance traveled by said buoyant element; and
a sensor for sensing a property of said fluid, said sensor being in communication with a processor above ground.

2. The fluid monitoring and sampling apparatus according to claim 1, further comprising a sample collecting vessel adapted to collect a sample of the fluid.

3. The fluid monitoring and sampling apparatus according to claim 1, wherein said sensor is adapted to sense at least one of a presence of dissolved solids in the fluid, a presence of oil in the fluid, a boundary between oil and the fluid, salinity, electrical conductivity, temperature, pressure, pH, viscosity, and density of the fluid.

4. The fluid monitoring and sampling apparatus according to claim 1, wherein said sensor is attached to said buoyant element.

5. The fluid monitoring and sampling apparatus according to claim 1, wherein said sensor is attached to a weight attached to said buoyant element.

6. The fluid monitoring and sampling apparatus according to claim 1, wherein said sensor is spooled down separately from said buoyant element on said filament.

7. The fluid monitoring and sampling apparatus according to claim 1, wherein said filament comprises an electrical wire that operates using single wire energy transmission techniques.
